(19) United States
(12) Patent Application Publication
(10) Pub. No.: US 2024/0151613 A1
(43) Pub. Date: May 9, 2024

(54) METHOD FOR EXTRACTION OF MYCOTOXINS FROM A SAMPLE

(71) Applicants: GOLDSWORTHY, Robert, Sydney, New South Wales (AU); GOLDSWORTH, Susan, Sydney, New South Wales (AU)

(72) Inventors: Robert John Goldsworthy, Bobs Farm NSW (AU); Susan Goldsworthy, Bobs Farm NSW (AU)

(21) Appl. No.: 18/281,405

(22) PCT Filed: Mar. 11, 2022
(86) PCT No.: PCT/AU2022/050204

§ 371 Date: Sep. 11, 2023

(30) Foreign Application Priority Data
Mar. 13, 2021 (AU) 2021900729
Aug. 1, 2021 (AU) 2021902370

(51) Int. Cl. G01N 1/38; G01N 1/28; G01N 1/40; G01N 33/569

(52) U.S. Cl. CPC G01N 1/38; G01N 1/286; G01N 33/56961; G01N 2001/386; G01N 2001/4061

(57) ABSTRACT

A method to extract a mycotoxin, or a plurality of mycotoxins, from a sample carrier into a solvent solution. The mycotoxin may have been an initially airborne mycotoxin initially captured by a filter that functions as the sample carrier. The method may generally include the steps of at least partially immersing or wetting the sample carrier within a solvent solution carried by a vessel; agitating the sample carrier within the solvent solution to extract at least some of the mycotoxin from the sample carrier; and removing the sample carrier from the vessel.

### RELATED CASES

This application claims priority from Australian provisional patent application nos. 2021900729 and 2021902370 filed on 13 Mar. 2021 and 1 Aug. 2021, respectively, the contents of which are incorporated by reference.

### BACKGROUND

The invention relates to a method for extraction of mycotoxins from a sample. In particular, the invention relates to a non-gravimetric method for extraction of airborne mycotoxins present in a sample collected from air and quantification of the extracted mycotoxins.

### BACKGROUND

Fungus such as mould may produce chemical by-products known as mycotoxins. Such mycotoxins may be hazardous to human health when such mycotoxins are present as a result of mould or water damage either currently or historically active within the built environment such as within a person's home.

Accordingly, methods and apparatuses have been developed to sample and test for mould within the built environment. Some methods may use a vacuum to collect mould spore samples from surfaces and then the samples may be tested to identify the mould type such as by crushing the spores to expose the DNA and identify the mould based on the DNA profile.

A problem with such identification of the mould species is that it does not specifically identify or allow quantification of the associated hazardous mycotoxins that may be present or to which a person may be exposed.

To more specifically quantify mycotoxins present in the indoor environment. A method to sample air from the indoor environment and collect mycotoxins from the air has been developed. WO/2021/081578 discloses such a method which includes collection of a mycotoxin sample from an air space of an indoor environment, the method may include conditioning air, such as with an air agitator, within an air space to provide conditioned air representative of air exposed to a person within the indoor environment and sampling the air such as by filtering the conditioned air at a height within the airspace further representative of air exposed to a person within the indoor environment so as to obtain the mycotoxin sample. The collected sample may then be extracted from the sample and analysed.

Air samples are different to sample extraction of a commodity (such as a mechanically captured sample) in that the air sample consists of finer, almost gaseous, particulates that have been captured within a sample collector such as a filter. Air samples may not require grinding, or any further filtration.

Existing methods of processing samples of commodities typically include steps, not appropriate for air samples, such as grinding a measured portion of the commodity in question. Another step, not appropriate for air samples, is the filtering of larger particulate matter from the ground commodity.

The invention disclosed herein seeks to overcome one or more of the above identified problems or at least provide a useful alternative.

### SUMMARY

In accordance with a first broad aspect there is provided, a method to extract a mycotoxin, or a plurality of mycotoxins, from a sample carrier into a solvent solution. The mycotoxin may have been an initially airborne mycotoxin initially captured by a filter that functions as the sample carrier. The method may generally include the steps of: at least partially immersing or wetting the sample carrier within a solvent solution carried by a vessel; agitating the sample carrier within the solvent solution to extract at least some of the mycotoxin from the sample carrier; and removing the sample carrier from the vessel.

In an aspect, the solvent solution includes alcohol.

In another aspect, the alcohol is methanol.

In yet another aspect, the solvent solution includes about 70% methanol and 30% water.

In yet another aspect, the water is deionised water.

In yet another aspect, the sample carrier is a filter.

In yet another aspect, the sample carrier is formed of foam.

In yet another aspect, the filter is formed of foam.

In yet another aspect, the step of immersing includes submersing the sample carrier within the solvent solution.

In yet another aspect, the vessel is a test tube.

In yet another aspect, the step of agitating the sample carrier within the solvent solution includes ultrasonication.

In yet another aspect, the step of removing the sample carrier from the vessel includes squeeze drying the sample carrier such that excess solvent solution is drained into the vessel.

In yet another aspect, the method further includes the step of processing the solvent solution to identify extracted mycotoxins.

In yet another aspect, the step of processing the solvent solution to identify extracted mycotoxins includes an enzyme-linked immunosorbent assay technique.

In accordance with a second broad aspect there is provided, a solution for processing including a mycotoxin extracted in accordance with a method as defined in any one of the previous claims.

In accordance with a third broad aspect there is provided, a method to extract initially airborne one or more mycotoxins from a filter in which the initially airborne one or more mycotoxins have been captured for processing, the method including: immersing the filter within a solvent solution carried by a vessel, the solvent solution including an alcohol; agitating the filter within the solvent solution to assist dissolving the one or more mycotoxins into the solvent solution; removing the sample carrier whilst drying the filter such that excess solvent solution is drained into the vessel; and processing the solvent solution to identify extracted mycotoxins.

In accordance with a fourth broad aspect there is provided, a method to extract initially airborne mycotoxins from a sample carrier in which the initially airborne mycotoxins have been captured for processing, the method including: immersing the sample carrier within a solvent solution carried by a vessel, the solvent solution including methanol and deionised water; agitating the sample carrier within the solvent solution using ultrasonication; removing the sample carrier whilst squeeze drying the sample carrier such that excess solvent solution is drained into the vessel; and processing the solvent solution to identify extracted mycotoxins using an enzyme-linked immunosorbent assay technique.

In accordance with a fifth broad aspect there is provided, a non-gravimetric method to extract one or more airborne mycotoxins from a sample carrier in which the one or more airborne mycotoxins have been collected by the sample carrier from air, the method including: immersing the sample carrier within a solvent solution carried by a vessel, the solvent solution including an alcohol to dissolve the one or more airborne mycotoxins into the solvent solution; agitating the sample carrier within the solvent solution using one or more of manual and ultrasonic agitation to extract the one or more airborne mycotoxins into the solvent solution; removing the sample carrier whilst at least partially drying the sample carrier such that excess solvent solution is drained into the vessel; and processing the solvent solution to identify one or more extracted airborne mycotoxins using an enzyme-linked immunosorbent assay technique.

In accordance with a sixth broad aspect there is provided, a method to determine a quantity of one or more airborne mycotoxins within a space extractable from a sample carrier in which the one or more airborne mycotoxins have been collected by the sample carrier from air, the method including: immersing the sample carrier within a solvent solution carried by a vessel, the solvent solution including methanol to dissolve the one or more airborne mycotoxins into the solvent solution; agitating the sample carrier within the solvent solution using one or more of manual and ultrasonic agitation to extract the one or more airborne mycotoxins into the solvent solution; removing the sample carrier whilst at least partially drying the sample carrier such that excess solvent solution is drained into the vessel; and processing the solvent solution to identify a quantity one or more extracted airborne mycotoxins using an enzyme-linked immunosorbent assay technique; determine based on the quantity a human exposure associated with the one or more airborne mycotoxins within the space.

In accordance with a seventh broad aspect there is provided, a method to determine a quantity of one or more airborne mycotoxins extractable from a sample carrier in which the one or more airborne mycotoxins have been collected by the sample carrier from air, the method including: immersing the sample carrier within a solvent solution carried by a vessel, the solvent solution including methanol to dissolve the one or more airborne mycotoxins into the solvent solution; agitating the sample carrier within the solvent solution using one or more of manual and ultrasonic agitation to extract the one or more airborne mycotoxins into the solvent solution; removing the sample carrier whilst at least partially drying the sample carrier such that excess solvent solution is drained into the vessel; and processing the solvent solution to identify a quantity one or more extracted airborne mycotoxins using an enzyme-linked immunosorbent assay technique; determine based on the quantity, a per cubic meter exposure associated with the one or more airborne mycotoxins within a given time period relative to a volume of air collected during a sampling period.

### BRIEF DESCRIPTION OF THE FIGURES

The invention is described, by way of non-limiting example only, by reference to the accompanying figures, in which;

FIG. 1 is a flow chart illustrating an example method for extraction of mycotoxins from a sample.

### DETAILED DESCRIPTION

Mycotoxins, including but not limited to, Aflatoxins B1, B2, G1, G2, Ochratoxin A, Fumonisin, Vomitoxin, Zearelenone, T-2, HT-2, may be targeted through the below described respiratory size collection methods and then undergo direct analysis via quantitative analysis to determine the airborne concentration of given mycotoxins within the breathable air and reported in ppb (Parts per Billion) relative to the volume of air collected during the sampling period.

Mycotoxins may be collected from air, such as air of the indoor environment, by a variety of techniques. WO/2021/081578, the contents of which are incorporated herein by reference, discloses such a method which includes collection of a mycotoxin sample from an air space of an indoor environment using one or more filters. The filters are disclosed as including, for example, a primary filter including a high-density foam, such as polyurethane foam, adapted to capture mycotoxins from the air drawn therethrough and a secondary filter that may be a PVC (Polyvinyl chloride) filter. Such filters provide a sample carrier (not shown) in which mycotoxins are captured and carried until extracted. Accordingly, there is a need for a method to extract the mycotoxins from the sample carrier for analysis and quantification.

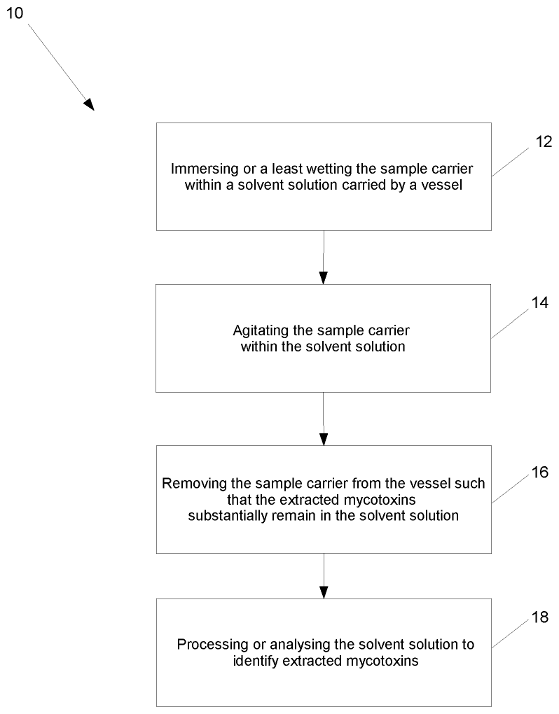

Referring to FIG. 1, there is shown a method 10 for the extraction of mycotoxins from a sample carrier or collector (not shown) for analysis and quantification. Such a mycotoxin is described herein as an initially airborne mycotoxin—which has been captured by the sample carrier or collector. The captured mycotoxins may still be airborne within air pockets or the like within the sample carrier or collector and thereby need to be removed from the air and sample carrier into a solution for processing.

The sample carrier may be a primary filter including a high-density foam, such as polyurethane foam (PUF), adapted to capture mycotoxins from the air drawn therethrough and a secondary filter that may be a PVC filter. The filters may have been used to collect the mycotoxins samples by drawing air through the primary filter and then the secondary filter. The polyurethane foam and PVC filter are collectively referred to here as “filters” that are used as the sample carrier or collector in which airborne mycotoxins have been collected for extraction. The initially airborne mycotoxins are located within air pockets or within or on the material structures of the filter and need to be extracted or “stripped” from the filter.

The method 10 includes at step 12, immersing or a least wetting the sample carrier within a solvent solution carried by a vessel to dissolve at least some or most of the captured mycotoxins into the solvent solution with permeates the sample carrier, at step 14 agitating the sample carrier within the solvent solution to extract some or most of the captured mycotoxins from the sample carrier into the solvent solution, and a step 16 removing the sample carrier from the vessel such that the extracted mycotoxins substantially remain in the solvent solution. Immersion assists here to ensure the airborne mycotoxins within the carrier medium, in this case the filter, become dissolved within the solvent solution. At step 18, once extracted, the method may further include processing or analysing the solvent solution to identify extracted mycotoxins.

Turning to the method 10 now in more detail, the solvent solution includes an alcohol, and preferably methanol. In this example, preferably, the solvent solution includes about 70% methanol and about 30% water. However, the ratios of ingredients may vary from about 60% to 80% methanol with 40% to 20% water, as appropriate. The water may be deionised water solvent solution.

It is noted that other suitable solvents could be used such as surfactants, namely, high alkaline surfactants. However, it has been found that methanol is advantageous in stripping and dissolving Mycotoxins. It is believed that a reason for this is that a Mycotoxin behaves like a lipoid and dissolves most suitability into alcohol, in particular methanol. Furthermore, alcohol, in particular methanol has been found to not degrade the sample carrier, such as the foam filter, which means that, for example, parts of the foam filters are not extracted into and contaminate the solvent solution.

The vessel may be a test tube such as a falcon tube, and, for example, about 10 millilitres of the solvent solution may be placed into the test tubes. The sample carrier, such as the foam filter, may be removed from any packaging or storage cartridge and then a user may, using tweezers or the like, grasp the foam and submerse within the solvent solution. It is noted that the vessel in which the foam and filter are to undergo the extraction process must exceed the circumference of the foam and filter to allow correct ultrasonification.

Agitating the sample carrier within the solvent solution may include manual agitation such as the user massaging the foam several times whilst holding in the solution until the sample carrier substantially saturated, being generally full of solution and remains submersed. The sample carrier, if formed of foam, will take on a more translucent appearance. If there are more than one sample carriers, then the process can be repeated for each of the sample carriers. The vessel may then be placed on a rack. Agitation assists to move and extract the airborne mycotoxins from within the sample carrier, in this case the filter.

In addition to manual agitation, the agitating the sample carrier within the solvent solution preferably includes ultrasonication for a time period, such as, but not limited to, 10 minutes. In other examples, the time period may be in the range of 1 to 20 minutes, 5 to 15 minutes or 9 to 11 minutes. This includes placing and removing the rack from an ultrasonication apparatus (not shown). Ultrasonication should occur on a medium setting, that being between 40 kHz and 200 kHz at a temperature of 20-24 degrees Celsius for a period, that is preferably, no less than 10 minutes.

The step of removing the sample carrier from the vessel preferably includes squeeze drying the sample carrier such that excess solvent solution is drained into the vessel. For example, a user may squeeze the sample carrier such as the foam filter using tweezers and massage it above the water line of the solution until the solution is substantially removed. If the sample carrier is a foam or filter the foam or filter may take on its original white appearance when the solution is extracted. The sample carrier may then be removed and discarded. The mycotoxins are now extracted into the solution. It has been found that up to about 80% or greater of captured mycotoxins may be extracted by this method into the solvent solution.

The solution may then be further processed to identify and quantify the extracted mycotoxins. After the extraction process and removal of the filter from the liquid, there may also be a further dilution step prior to processing the sample using ELISA (Enzyme-Linked Immunosorbent Assay). The dilution step may involve 1 part sample and 3 parts deionized water. For example, using a pipettor, 1 ml amount of the solution may be transferred into a 7 ml tube and 3 mls of deionized water may be added to form a processing sample.

The processing sample is now ready for processing by a non-gravimetric enzyme-linked immunosorbent assay technique for the desired mycotoxins and the identified mycotoxins can be reported such as in a cubic meter format, more specifically, a parts per cubic meter exposure within a given time period relative to the volume of air collected during the sampling period. Each space, such as a home or workplace, has different size rooms and each occupant uses a space differently and for longer and shorter times. For example, if 1-hour sample is taken within a room, that is the sample that is analysed (allowing our dilution rates etc). If that sample is taken by a pump at a rate of a round figure of, say, 100/litres per hour, then it is known to multiply the result by 10 to report in a cubic meter format (1,000 litres=1 cubic meter).

Advantageously, there has been disclosed a method for extraction of mycotoxins from a sample into a solvent solution which provides increased yield and targeting of mycotoxins over other methods such as the use of separation columns both specific and non-specific commonly used in food and feed extraction methods. In particular, the method allows for extraction of airborne mycotoxins present in a sample collected from air and analysis and quantitation of such collected airborne mycotoxins. The method enables the results to be reported as a parts per cubic meter exposure within a given time period relative to the volume of air collected during the sampling period which allows the human exposure to mycotoxins to be determined and subsequent test results to follow a repeatable process for on-going comparison.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any known matter or any prior publication is not, and should not be taken to be, an acknowledgment or admission or suggestion that the known matter or prior art publication forms part of the common general knowledge in the field to which this specification relates.

While specific examples of the invention have been described, it will be understood that the invention extends to alternative combinations of the features disclosed or evident from the disclosure provided herein.

Many and various modifications will be apparent to those skilled in the art without departing from the scope of the invention disclosed or evident from the disclosure provided herein.

1-19. (canceled)

20. A non-gravimetric method to extract one or more airborne mycotoxins from a sample carrier in which the one or more airborne mycotoxins have been collected by the sample carrier from air, the method including:
immersing the sample carrier within a solvent solution carried by a vessel, the sample carrier including a primary filter formed of polyurethane high-density foam and a secondary filter formed of polyvinyl chloride, the solvent solution including about 70% methanol and about 30% water to separate the one or more airborne mycotoxins from the primary filter and the secondary filter;
manually agitating at least the primary filter while holding the primary filter in the solvent solution until the primary filter is saturated;
further agitating the primary filter and secondary filter within the solvent solution using ultrasonic agitation to extract the one or more airborne mycotoxins from the primary filter and the secondary filter into the solvent solution;
removing the primary filter and secondary filter from the vessel while at least partially squeeze drying the primary filter and the secondary filter such that excess solvent solution is drained into the vessel; and
processing the solvent solution to identify one or more extracted airborne mycotoxins using an enzyme-linked immunosorbent assay technique.

21. The method according to claim 20, wherein the water is deionized water.

22. The method according to claim 20, wherein the vessel is a test tube.

23. The method according to claim 20, wherein agitating the sample carrier within the solvent solution includes ultrasonication.

24. The method according to claim 20, wherein removing the sample carrier from the vessel includes squeeze drying the sample carrier such that excess solvent solution is drained into the vessel.

25. The method according to claim 20 further including:
processing the solvent solution to identify the extracted mycotoxin.

26. The method according to claim 25, wherein processing the solvent solution to identify the extracted mycotoxin includes an enzyme-linked immunosorbent assay technique.

27. A non-gravimetric method to extract one or more airborne mycotoxins from a sample carrier in which the one or more airborne mycotoxins have been collected by the sample carrier from air, the method including:
immersing the sample carrier within a solvent solution carried by a vessel, the solvent solution including an alcohol to dissolve the one or more airborne mycotoxins into the solvent solution;
agitating the sample carrier within the solvent solution using one or more of manual and ultrasonic agitation to extract the one or more airborne mycotoxins into the solvent solution;
removing the sample carrier whilst at least partially drying the sample carrier such that excess solvent solution is drained into the vessel; and
processing the solvent solution to identify one or more extracted airborne mycotoxins using an enzyme-linked immunosorbent assay technique.

28. A method to determine a quantity of one or more airborne mycotoxins within a space extractable from a sample carrier in which the one or more airborne mycotoxins have been collected by the sample carrier from air, the method including:
immersing the sample carrier within a solvent solution carried by a vessel, the solvent solution including methanol to dissolve the one or more airborne mycotoxins into the solvent solution;
agitating the sample carrier within the solvent solution using one or more of manual and ultrasonic agitation to extract the one or more airborne mycotoxins into the solvent solution;
removing the sample carrier while at least partially drying the sample carrier such that excess solvent solution is drained into the vessel; and
processing the solvent solution to identify a quantity one or more extracted airborne mycotoxins using an enzyme-linked immunosorbent assay technique; and
determining based on the quantity a human exposure associated with the one or more airborne mycotoxins within the space.
